(19) United States
(12) Patent Application Publication
(10) Pub. No.: US 2026/0000541 A1
(43) Pub. Date: Jan. 1, 2026

(54) OPHTHALMIC LASER TREATMENT APPARATUS AND TREATMENT LASER LIGHT IRRADIATION UNIT

(71) Applicant: NIDEK CO.,LTD., Gamagori-shi (JP)

(72) Inventors: Takahiko NAKANO, Gamagori-shi (JP); Tomoki NISHIMURA, Gamagori-shi (JP); Joji SASAKI, Gamagori-shi (JP)

(21) Appl. No.: 19/246,686

(22) Filed: Jun. 24, 2025

(30) Foreign Application Priority Data
Jun. 28, 2024 (JP) 2024-105128
Jun. 28, 2024 (JP) 2024-105129

(51) Int. Cl. A61F 9/008

(52) U.S. Cl. CPC A61F 9/00821; A61F 2009/00863; A61F 2009/00897

(57) ABSTRACT

An ophthalmic laser treatment apparatus includes a laser treatment apparatus main body that irradiates a first treatment laser light onto a patient's eye via an objective lens; a treatment laser light irradiation unit mounted above the objective lens on the laser treatment apparatus main body. The treatment laser light irradiation unit irradiates a second treatment laser light, different from the first treatment laser light, onto the patient's eye via a final mirror disposed at a predetermined irradiation position in front of the objective lens. The ophthalmic laser treatment apparatus further includes a movement mechanism that moves the final mirror between the irradiation position and a predetermined retracted position outside an optical path of the first treatment laser light. The movement mechanism laterally moves the final mirror from the irradiation position to the retracted position relative to the objective lens.

### CROSS REFERENCE TO RELATED APPLICATION

This application is based on, and claims the benefit of priority from Japanese Patent Application No. 2024-105128 on Jun. 28, 2024 and Japanese Patent Application No. 2024-105129 on Jun. 28, 2024. The entire disclosure of the above applications is incorporated herein by reference.

### TECHNICAL FIELD

The present disclosure relates to an ophthalmic laser treatment apparatus for irradiating a treatment laser light onto a patient's eye and a combinable treatment laser light irradiation unit attachable to the ophthalmic laser treatment apparatus.

### BACKGROUND ART

Conventional ophthalmic laser treatment apparatuses are known that irradiate a treatment laser light onto a patient's eye while observing the eye tissue through a binocular microscope. In such apparatuses, a combined laser treatment apparatus has been proposed (e.g., Patent Document 1: JP H07-016253 A) where a treatment laser irradiation unit, equipped with a second treatment laser irradiation optical system emitting a second treatment laser light (e.g., photocoagulation laser) for a therapeutic purpose different from a first treatment laser light (e.g., YAG laser, SLT laser) irradiated via an objective lens of a first treatment laser irradiation optical system and an observation optical system, is mounted. In this apparatus, a final mirror reflecting the second treatment laser light toward the patient's eye is movable (insertable and retractable) in front of the objective lens of the first treatment laser irradiation optical system. During irradiation of the second treatment laser light, the final mirror is moved to an irradiation position in front of the objective lens, and during irradiation of the first treatment laser light, the final mirror is moved to a retracted position away from the objective lens.

Regarding the final mirror's movement mechanism, conventional devices, for example, rotate the final mirror about a rotation axis perpendicular to the optical axis of the objective lens to move it to an upper retracted position. Another known laser treatment apparatus (e.g., Patent Document 2: JP 2011-212349 A) comprises a treatment laser irradiation unit attached to a binocular microscope, which scans a treatment laser light (e.g., photocoagulation laser) on the patient's eye tissue via a final mirror disposed in front of the microscope's objective lens. Here, the operator adjusts the irradiation position of the treatment laser light by mechanically manipulating the final mirror.

### SUMMARY

In combined ophthalmic laser treatment apparatuses, further improvements are needed. For example, when a barrel holding optical components of the second treatment laser irradiation optical system is positioned close to the upper side of the objective lens, rotating the final mirror upward for retraction risks interference with the barrel. Similarly, if components of an illumination unit are arranged in front of the objective lens, upward rotation of the final mirror may interfere with these components. Additionally, when a treatment laser irradiation unit with a scanning part is mounted on a laser treatment apparatus main body, mechanically adjusting the final mirror's position is challenging.

The present disclosure aims to provide an ophthalmic laser treatment apparatus and irradiation unit that appropriately moves the final mirror while avoiding interference. A second aim is to enable precise irradiation of the treatment laser light onto the patient's eye.

In a first aspect of the present disclosure, an ophthalmic laser treatment apparatus includes:
a laser treatment apparatus main body configured to irradiate a first treatment laser light onto a patient's eye via an objective lens;
a treatment laser light irradiation unit mounted above the objective lens on the laser treatment apparatus main body, the treatment laser light irradiation unit configured to irradiate a second treatment laser light, different from the first treatment laser light, onto the patient's eye via a final mirror disposed at a predetermined irradiation position in front of the objective lens; and
a movement mechanism configured to move the final mirror between the irradiation position and a predetermined retracted position outside an optical path of the first treatment laser light.

The movement mechanism is configured to laterally move the final mirror from the irradiation position to the retracted position relative to the objective lens.

In a second aspect of the present disclosure, an ophthalmic laser treatment apparatus includes:
a laser treatment apparatus main body configured to irradiate a first treatment laser light onto a patient's eye via an objective lens;
a treatment laser irradiation unit mounted on the laser treatment apparatus main body, the treatment laser irradiation unit comprising a second treatment laser irradiation optical system with a scanning part configured to scan a second treatment laser light, different from the first treatment laser light, onto a patient's eye tissue via a final mirror disposed at a predetermined irradiation position in front of the objective lens;
a movement mechanism configured to move the final mirror between the irradiation position and a retracted position outside an optical path of the first treatment laser light;
an operation unit configured to input an operation signal from an operator; and a control unit.

The second treatment laser irradiation optical system is configured to form a plurality of spots arranged in a predetermined scan pattern via the scanning part, the operation unit is further configured to perform both (i) a manipulator function of inputting a movement signal for two-dimensionally displacing an irradiation position of the second treatment laser light on the patient's eye tissue and (ii) a pattern setting function of setting the scan pattern.

The control unit is configured to control the scanning part based on signals of the manipulator function and the pattern setting function that are input from the operation unit.

In a third aspect of the present disclosure, a treatment laser irradiation unit is mounted above an objective lens of a laser treatment apparatus main body configured to irradiate a first treatment laser light onto a patient's eye via the objective lens. The treatment laser irradiation unit includes:
a second treatment laser irradiation optical system configured to irradiate a second treatment laser light, different from the first treatment laser light, onto the patient's eye via a final mirror disposed at a predetermined irradiation position in front of the objective lens; and
a movement mechanism configured to move the final mirror between the irradiation position and a retracted position outside an optical path of the first treatment laser light.

The movement mechanism is configured to move the final mirror to the retracted position by laterally moving the final mirror at the irradiation position relative to the objective lens.

### BRIEF DESCRIPTION OF DRAWINGS

FIG. 1 is a diagram illustrating an overall configuration of the ophthalmic laser treatment apparatus.

FIG. 2 is a diagram illustrating optical and control systems.

FIG. 3 is a top view of the observation optical system.

FIG. 4 is a right-side sectional view of the treatment laser irradiation unit.

FIG. 5 is a front view of the unit (a: irradiation position, b: retracted position).

FIG. 6 is a top view of the unit's internal structure.

FIG. 7 is a perspective view of the mirror movement mechanism.

FIG. 8 is a final mirror position detection mechanism.

FIG. 9 is a control box screens during operation.

FIG. 10 shows examples of laser spot scan patterns.

FIG. 11 is a diagram illustrating 3D mouse configuration and input signals.

### DETAILED DESCRIPTION OF THE PREFERRED EMBODIMENTS

### [Overview]

A typical embodiment will be described with reference to the drawings. It should be noted that the items classified in < > below can be used independently or in combination.

For example, an ophthalmic laser treatment apparatus (e.g., apparatus 1) comprises a laser treatment apparatus main body (e.g., main body 100), a treatment laser light irradiation unit (e.g., unit 200), and a movement mechanism (e.g., mechanism 250). The apparatus may further comprise a control unit (e.g., control unit 50).

The apparatus comprises a first treatment laser irradiation optical system (e.g., system 110G) configured to irradiate a first treatment laser light onto the patient's eye via an objective lens (e.g., lens 125). The treatment laser irradiation unit is mounted above the objective lens on the main body. Here, “above the objective lens” refers to the upper side relative to the optical axis of the objective lens or the patient's head side during irradiation of the first treatment laser light.

The treatment laser irradiation unit comprises a second treatment laser irradiation optical system (e.g., system 200G) configured to irradiate a second treatment laser light, different from the first, onto the patient's eye via a final mirror (e.g., mirror 228) disposed at a predetermined irradiation position in front of the objective lens (on the patient's eye side). The irradiation position of the final mirror is set on the optical axis of the objective lens, and the reference optical axis of the second treatment laser light reflected by the final mirror substantially coincides with the optical axis of the objective lens.

The second treatment laser light serves a therapeutic purpose distinct from the first treatment laser light. While the wavelengths of the first and second treatment laser lights may differ, they may also share the same wavelength if their therapeutic effects differ.

For example, the ophthalmic laser treatment apparatus may include an observation optical system (e.g., an observation optical system 140G). The observation optical system is configured to observe the patient's eye via the objective lens. Furthermore, for example, the observation optical system may include a microscope (e.g., microscope 141) with binocular eyepieces. Additionally, for example, the ophthalmic laser treatment apparatus may include an illumination optical system (e.g., illumination optical system 130G). For example, the illumination optical system is configured to project illumination light onto the patient's eye via a reflective member, exemplified by a split mirror (e.g., split mirror 136), disposed on the patient's eye side relative to the objective lens. In this case, the predetermined irradiation position of the final mirror may be set between the objective lens and the reflective member of the illumination optical system. This configuration allows irradiation of the second therapeutic laser beam without increasing the working distance during irradiation of the second therapeutic laser beam relative to the working distance during irradiation of the first therapeutic laser beam.

For example, the movement mechanism is configured to move the final mirror between a predetermined irradiation position in front of the objective lens and a predetermined retracted position outside the optical path of the first treatment laser light (i.e., the optical path of the objective lens). Specifically, the movement mechanism is configured to displace the final mirror, placed at the irradiation position, laterally relative to the objective lens to the retracted position. This configuration allows the final mirror to be appropriately moved while avoiding interference with components of the treatment laser irradiation unit. For instance, even if the barrel of the second treatment laser irradiation optical system is positioned close to the objective lens of the first treatment laser irradiation optical system above the final mirror, the final mirror can be retracted from the front of the objective lens without interfering with the barrel. Similarly, if a component of the illumination unit (e.g., a reflective member of the illumination optical system) is arranged in front of the objective lens, the final mirror can be moved appropriately while avoiding interference with that component.

Also, in other words, for example, the movement mechanism is configured to move the final mirror, which is placed at the predetermined retracted position, to the irradiation position between the objective lens and the reflective member of the illumination optical system. Note that the lateral direction relative to the objective lens refers to the lateral direction in a state where the therapeutic laser beam irradiation unit is arranged above the objective lens. Furthermore, the lateral direction relative to the objective lens may be an obliquely upward or obliquely downward lateral direction relative to the optical axis of the objective lens.

For example, the movement mechanism may be configured to move the final mirror, which is placed at a predetermined irradiation position, to a predetermined retracted position by pivoting it about a rotation axis (e.g., rotation axis R1) that is parallel to the optical axis of the objective lens. By adopting a rotation mechanism as the movement mechanism, the final mirror can be appropriately moved in a lateral direction that avoids interference with a lens barrel of the second therapeutic laser irradiation optical system located above the final mirror, without complicating or enlarging the structure of the movement mechanism. In this case, the final mirror is moved in a lateral direction that is obliquely upward or obliquely downward from the irradiation position.

For example, the movement mechanism comprises an arm (e.g., arm 265) rotatably attached about the rotation axis, supporting the final mirror at its tip. The final mirror is laterally rotated when the arm rotates about the rotation axis. The arm extends above the final mirror when the mirror is at the irradiation position.

For example, the rotation axis may be positioned above the objective lens, and the predetermined retracted position may be set above the objective lens. In this case, the movement mechanism may be configured to move the final mirror, which is placed at the predetermined irradiation position, to the predetermined retracted position in a lateral direction relative to the objective lens and above the objective lens. Thereby, since the final mirror is retracted above the objective lens, the final mirror also moves out of an observation optical path of the observation optical system. Consequently, during treatment using the first therapeutic laser beam, the apparatus can be used in the same manner as when the therapeutic laser beam irradiation unit is not mounted.

The rotation axis is preferably aligned directly above (or substantially above) the objective lens's optical axis. This positions the retracted final mirror laterally adjacent to the barrel (e.g., barrel 220a) of the treatment laser irradiation unit.

The treatment laser irradiation unit may comprise a vertical adjustment mechanism (e.g., mechanism 270A) configured to adjust the tilt angle of the final mirror in the vertical direction. The vertical adjustment mechanism moves synchronously with the final mirror via the movement mechanism, avoiding complexity and an increase in size.

The treatment laser irradiation unit may comprise a detection sensor (e.g., final mirror position detection mechanism 290). The detection sensor is configured to detect whether the final mirror is positioned at the irradiation position or the retracted position. When the detection sensor is provided, the control unit controls irradiation of the first treatment laser light from the first treatment laser irradiation optical system and the second treatment laser light from the second treatment laser irradiation optical system based on the detection result. If the detection sensor detects the final mirror at the retracted position, the control unit disables the second treatment laser light and enables the first treatment laser light. If the detection sensor detects the final mirror at the irradiation position, the control unit disables the first treatment laser light and enables the second treatment laser light. If the final mirror is detected at neither position, the control unit disables both lasers. This prevents erroneous irradiation of the first and second treatment laser lights.

The ophthalmic laser treatment apparatus may comprise a trigger signal input mechanism (e.g., foot switch 54) for initiating laser irradiation. The trigger signal input mechanism may be used for both the first and second treatment laser lights. When the detection sensor detects the final mirror at the retracted position, the control unit activates the first treatment laser light in response to the trigger signal input. When the detection sensor detects the final mirror at the irradiation position, the control unit activates the second treatment laser light in response to the trigger signal input.

For example, the treatment laser irradiation unit may comprise a housing compartment (e.g., housing 280) located lateral to the barrel (e.g., barrel 220a) of the second treatment laser irradiation optical system positioned above the final mirror. This housing accommodates the final mirror when moved to the retracted position within a cover. This configuration prevents accidental operator contact with the final mirror during treatment using the first treatment laser light and reduces dust or contamination on the mirror surface.

The ophthalmic laser treatment apparatus may further comprise, in addition to the laser treatment apparatus main body, treatment laser irradiation unit, and movement mechanism: an operation unit (e.g., 3D mouse 53), and a control unit (e.g., control unit 50). The second treatment laser irradiation optical system of the treatment laser irradiation unit comprises a scanning part configured to scan the second treatment laser light, different from the first treatment laser light, on the patient's eye tissue. The second treatment laser light is irradiated onto the patient's eye via the final mirror positioned at the predetermined irradiation location in front of the objective lens. The second treatment laser irradiation optical system is configured to form multiple spots arranged in a predetermined scan pattern by scanning the second treatment laser light with the scanning part.

The operation unit is configured to input operation signals from the operator. For example, when combined with the scanning part, the operation unit may perform both: a manipulator function to input movement signals for two-dimensionally displacing the irradiation position of the second treatment laser light on the patient's eye tissue via the final mirror, and a pattern-setting function to define the scan pattern. The control unit is configured to control the scanning part based on signals from the manipulator and pattern-setting functions input via the operation unit. This enables precise irradiation of the treatment laser light onto the patient's eye even when the treatment laser irradiation unit comprises a scanning part. Specifically, even with a movement mechanism for displacing the final mirror in front of the objective lens, a single operation unit allows both (i) two-dimensional adjustment of the second treatment laser's irradiation position on the tissue and (ii) configuration of the scan pattern. Consequently, the usability of the ophthalmic laser treatment apparatus is significantly enhanced.

The operation unit may comprise a single operation member capable of both: a first operation (e.g., tilt operation) for two-dimensional displacement, and a second operation (e.g., slide operation), distinct from the first operation, for two-dimensional displacement. In this configuration, the control unit accepts a signal from either the first or second operation as the signal of the manipulator function (for two-dimensionally moving the irradiation position of the second treatment laser light on the patient's eye tissue) and controls the scanning part accordingly. In other words, the manipulator function is assigned to both: the first operation (two-dimensional displacement via tilt), and the second operation (two-dimensional displacement via slide). This allows operators who struggle with the first operation to use the second operation, and vice versa, improving the operation unit's usability. The apparatus may further comprise a signal assignment mechanism to assign the manipulator function to multiple operation signals input via the operation unit.

Additionally, the operation member of the operation unit may be capable of a third operation distinct from the first and second operations. When a signal from this third operation is input from the operation unit, the control unit may accept it as an origin return signal for the manipulator function, thereby controlling the scanning part to return the irradiation position of the second treatment laser light to the origin. This further enhances the usability of the ophthalmic laser treatment apparatus

The pattern-setting function may be configured to enable at least one of a first setting for rotating the scan pattern, a second setting for adjusting the interval (space) between adjacent spots forming the scan pattern, and a third setting for modifying the shape (e.g., square, triangle, arc) of arrangement of the spots or the number of spots in the scan pattern. In this case, the pattern-setting function's operation signals may be input via the operation member of the operation unit using actions distinct from those for the manipulator function. This allows the same operation unit, which inputs manipulator function signals, to also modify the predetermined scan pattern of the second treatment laser light spots according to the treatment site.

For example, the operation member of the operation unit may be configured to be operable by gripping with the hand or fingers while the operator observes the patient's eye, and to allow operation without the operator needing to visually confirm the controls. This enhances the usability of the ophthalmic laser treatment apparatus and enables the treatment laser light to be appropriately irradiated onto the patient's eye.

For example, when the ophthalmic laser treatment apparatus comprises the aforementioned observation optical system (e.g., observation optical system 140G), the final mirror may be configured to: ensure at least 80% of the observation field of view is maintained when positioned at the irradiation location; and have a size that permits irradiation of the second treatment laser light in the predetermined scan pattern. This allows the operator to observe the patient's eye while appropriately irradiating the treatment laser light scanned by the scanning part.

Furthermore, when the second treatment laser irradiation optical system comprises a scanning part (e.g., scanning part 230) and the apparatus is equipped with the aforementioned illumination optical system, the predetermined irradiation position of the final mirror may be set between the objective lens and the reflective member (e.g., split mirror 136) of the illumination optical system. This configuration allows irradiation of the second treatment laser light without increasing the working distance during its use compared to the working distance during irradiation of the first treatment laser light.

### Embodiment

An embodiment of the present disclosure will be described below with reference to the drawings.

### <Overall Configuration>

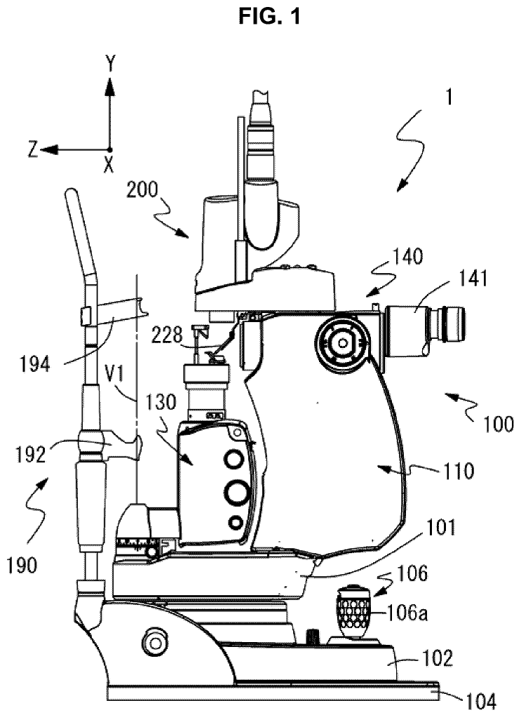

FIG. 1 illustrates the overall configuration of the ophthalmic laser treatment apparatus 1. The apparatus 1 comprises a laser treatment apparatus main body 100 and a treatment laser irradiation unit 200. The treatment laser irradiation unit 200 is detachably mounted on the main body 100. FIG. 1 shows a left-side view of the main body 100 as seen from the operator's perspective.

The laser treatment apparatus main body 100 comprises: a first treatment laser irradiation unit 110, an illumination unit 130, an observation unit 140, and a face support unit 190 for stabilizing the patient's head. The face support unit 190 comprises a chin rest 192 and a forehead rest 194. In FIG. 1, directions are defined as follows relative to the operator:
Left-right: X-direction,
Up-down: Y-direction,
Front-back: Z-direction.

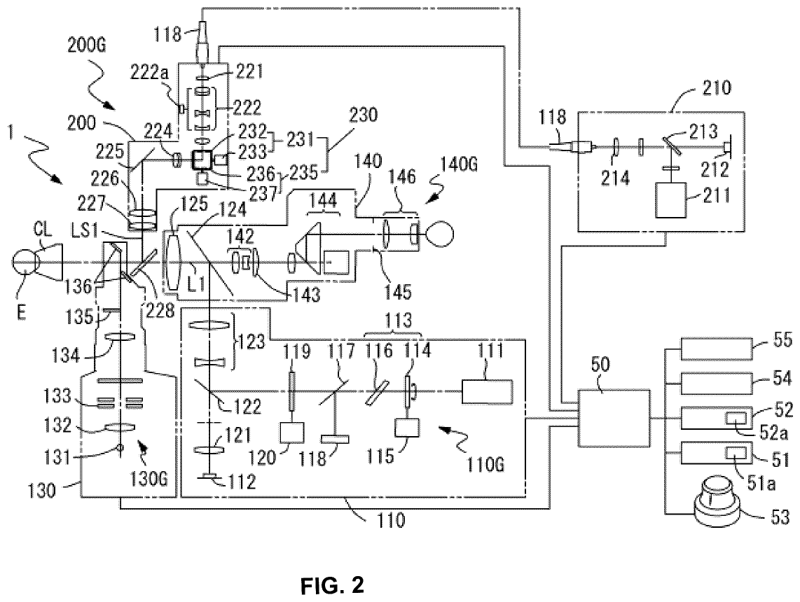

The first treatment laser irradiation unit 110 is configured to irradiate a first treatment laser light onto the patient's eye via an objective lens 125 (see FIG. 2). The unit 110 is mounted on a Y-stage 101, which is movable in the Y-direction relative to an XZ-stage 102. The XZ-stage 102 is movable in the X- and Z-directions on a table 104. A joystick 106 on the XZ-stage 102 allows the operator to move the first treatment laser irradiation unit 110 together with the XZ-stage 102 in the XZ-directions. A rotation knob 106a on the joystick 106 controls vertical (Y-direction) movement via a known mechanism.

Additionally, the illumination unit 130 is configured to project illumination light for observation onto the patient's eye. The illumination unit 130 is mounted on the Y-movable stage 101 and is moved in the XYZ directions together with the first therapeutic laser irradiation unit 110. Furthermore, the illumination unit 130 is rotatably mounted on the Y-movable stage 101 about a V1 axis (see FIG. 1) set on the front side of the Y-movable stage 101, the V1 axis extending in the vertical direction, such that the illumination unit 130 is rotatable in the left-right direction. Thereby, the left-right direction angle of the illumination light projected from the illumination unit 130 onto the patient's eye can be changed arbitrarily.

The observation unit 140, equipped with a binocular microscope 141, allows the operator to observe the patient's eye. It is mounted above the first treatment laser irradiation unit 110 and moves in the XYZ-directions with the unit 110.

The treatment laser irradiation unit 200 is configured to irradiate a second treatment laser light, different from the first, onto the patient's eye via a final mirror 228 positioned at a predetermined irradiation location in front of the objective lens 125.

The apparatus further comprises: a control unit 50, a first control box 51 for the main body 100, a second control box 52 for the unit 200, a 3D mouse 53 (operation unit for the unit 200), and a foot switch 54 (trigger signal input mechanism). The 3D mouse 53 may be a commercially available device.

### <Optical System>

FIG. 2 illustrates the optical and control systems of the ophthalmic laser treatment apparatus 1. The laser treatment apparatus main body 100 comprises: a first treatment laser irradiation optical system 110G (in unit 110), an illumination optical system 130G (in unit 130), and an observation optical system 140G (in unit 140). The treatment laser irradiation unit 200 comprises a second treatment laser irradiation optical system 200G.

The second treatment laser light from system 200G achieves a therapeutic effect distinct from the first treatment laser light from system 110G.

### <First Treatment Laser Irradiation Optical System>

The first treatment laser irradiation optical system 110G comprises: a treatment laser light source 111, an aiming light source 112, an energy adjustment unit 113, a beam splitter 117, a photodetector 118, a safety shutter 119, a collimator lens 121, a dichroic mirror 122, an expander lens 123, a dichroic mirror 124, and an objective lens 125.

The treatment laser light source 111 emits a therapeutic laser beam for treating the patient's eye tissue. In this embodiment, the source 111 uses an Nd: YAG (neodymium-doped yttrium aluminum garnet) crystal to emit infrared laser light (wavelength: 1064 nm). A wavelength conversion element (not shown) converts this to visible laser light (wavelength: 532 nm).

The aiming light source 112 emits an aiming laser beam (hereafter “aiming light”) indicating the irradiation position of the treatment laser light. In this embodiment, the aiming light source 112 emits visible red laser light (wavelength: 635 nm).

The energy adjustment unit 113 adjusts the energy of the treatment laser light irradiated onto the patient's eye tissue. It comprises: a half-wave plate 114 rotated by a motor 115 about the laser's optical axis and a polarizer 116 positioned at the Brewster angle. The combination of the half-wave plate and polarizer modulates the laser energy.

The beam splitter 117 reflects a portion of the treatment laser light toward the photodetector 118, which measures the laser energy upon receiving the reflected treatment laser light. The safety shutter 119, driven by a shutter driving member (e.g., a solenoid 120), moves into or out of the laser's optical path to block irradiation when needed.

The collimator lens 121 collimates the aiming light emitted from the aiming light source 112 into a parallel beam. The dichroic mirror 122 reflects the treatment laser light while transmitting the aiming light, coaxially aligning their optical axes and combining the beams. The expander lens 123 enlarges the beam diameter of the laser light (treatment laser light and aiming light). The expanded laser light is reflected by the dichroic mirror 124 and transmitted through the objective lens 125. The laser light transmitted through the objective lens 125 travels along the optical axis L1 of the objective lens and is irradiated onto the tissue of the patient's eye via a contact lens CL attached to the eye. The dichroic mirror 124 is configured to predominantly reflect the wavelength of the treatment laser light reflected by the patient's eye, minimizing backscatter into the operator's view. The irradiation optical system 110G may comprise additional configurations for adjusting the spot size of the laser light irradiated onto the tissue.

### <Illumination Optical System>

The illumination optical system 130G comprises an illumination light source 131 and, in order from the light source side, a condenser lens 132, a slit 133, a projection lens 134, a correction lens 135, and a split mirror 136. Illumination light emitted from the illumination light source 131 is projected onto the patient's eye E via the condenser lens 132, slit 133, projection lens 134, correction lens 135, and split mirror 136.

### <Observation Optical System>

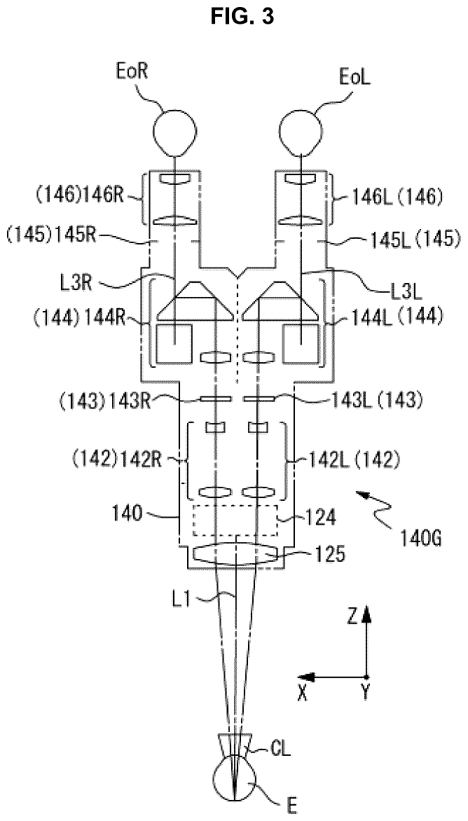

FIG. 3 is a top view of the observation optical system 140G. As shown in FIGS. 2 and 3, the observation optical system 140G is an observation means for allowing the operator to observe the patient's eye and comprises an optical axis L3 (see FIG. 3). As shown in FIG. 3, the observation optical system 140G comprises an optical axis L3R for presenting an observation image to the operator's right eye EoR and an optical axis L3L for presenting an observation image to the operator's left eye EoL. The observation optical system 140G is used as a binocular microscope 141. The observation optical system 140G comprises an objective lens 125 shared with the first treatment laser irradiation optical system 110G, a variable magnification optical system 142 (142R, 142L), protective filters 143 (143R, 143L), erecting prism groups 144 (144R, 144L), field diaphragms 145 (145R, 145L), and eyepiece lenses 146 (146R, 146L), among others. The operator can observe the observation site of the patient's eye, the aiming light spot (i.e., the return light of the aiming light reflected by the patient's eye), and so on by looking through the eyepiece lenses 146.

In this embodiment, the observation surface (object plane) at the tip of the objective lens 125 and the field diaphragms 145 inside the apparatus are optically conjugate via the objective lens. The observation image of the patient's eye is formed as an aerial image at the diaphragms. The magnification is adjusted via the variable magnification optics 142, with an encoder (not shown) tracking the magnification setting.

### <Second Treatment Laser Irradiation Optical System>

The second treatment laser irradiation optical system 200G comprises a treatment laser light source 211 and an aiming light source 212 provided in a laser light source unit 210. For example, the treatment laser light source 211 emits a second treatment laser beam in the visible wavelength range (e.g., 532 nm (green), 577 nm (yellow), 647 nm (red), etc.) used for photocoagulation treatment of the fundus of the patient's eye. In this embodiment, the treatment laser light source 211 is controlled to selectively emit either of two types (green/yellow) of treatment laser beams from the treatment laser light source 211. The aiming light source 212 emits an aiming light for allowing the operator to recognize the planned irradiation position of the second treatment laser beam. For example, a wavelength of 670 nm (red) is used as the wavelength of the aiming light. The second treatment laser beam from the treatment laser light source 211 and the aiming light from the aiming light source 212 are combined by a beam combiner 213, focused by a focusing lens 214, and incident on the entrance end face of an optical fiber 218.

The second treatment laser beam (and aiming light) emitted from the exit end face of the optical fiber 218 passes through a lens 221, a zoom lens 222, a scanning part (scanning optical system) 230, a relay lens 224, a mirror 225, a collimator lens 226, and an objective lens (imaging lens) 227, and is reflected by the final mirror 228, thereby being irradiated onto the tissue of the patient's eye via a contact lens CL. By moving the zoom lens 222 in the optical axis direction, the spot size of the second treatment laser beam irradiated onto the tissue of the patient's eye can be changed. The movement position of the zoom lens 222 is detected by an encoder 222a.

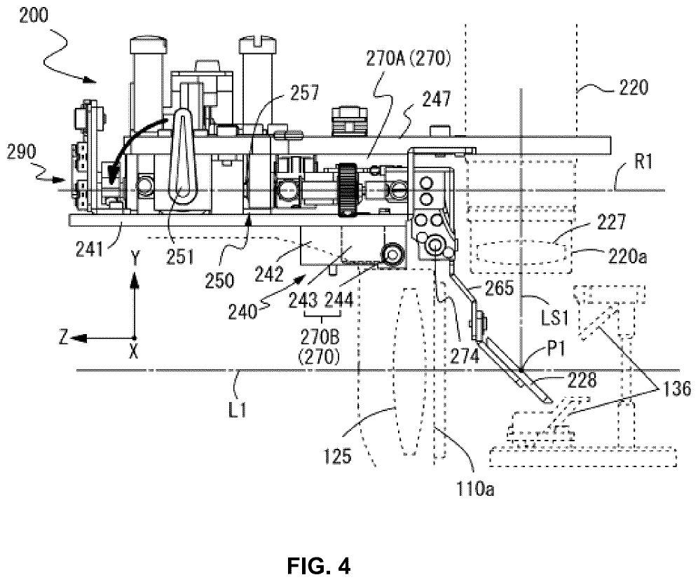

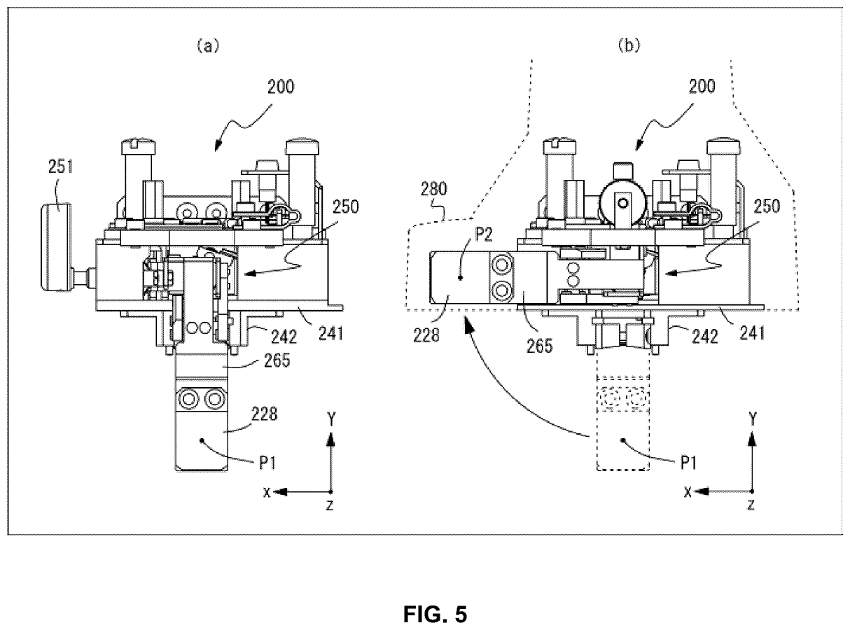

The final mirror 228 is arranged at an inclined position on the optical axis LS1 of the objective lens 227 and at a predetermined irradiation position P1 (see FIGS. 4-7) in front of the objective lens 125 (on the patient's eye side) of the first treatment laser irradiation optical system 110G. The irradiation position of the final mirror 228 is a position on the optical axis of the objective lens 125, and the reference optical axis of the second treatment laser beam reflected by the final mirror 228 and irradiated onto the patient's eye coincides (or substantially coincides) with the optical axis L1 of the objective lens 125. The final mirror 228 is moved between the irradiation position P1 in front of the objective lens 125 and a predetermined retracted position P2 (see FIG. 5) outside the irradiation optical path of the first treatment laser beam of the first treatment laser irradiation optical system 110G by a mirror movement mechanism 250 described later.

The scanning part 230 is configured to two-dimensionally scan the spot (irradiation position) of the second treatment laser beam on the tissue of the patient's eye (e.g., on the fundus). For example, the scanning part 230 comprises a first galvanometer mirror 231 as an example of a scanner mirror and a second galvanometer mirror 235. The first galvanometer mirror 231 comprises a mirror 232 and an actuator 233 and scans the second treatment laser beam reflected by the final mirror 228 in the left-right direction (X direction). The second galvanometer mirror 235 comprises a mirror 236 and an actuator 237 and scans the second treatment laser beam reflected by the final mirror 228 in the up-down direction (Y direction). Thus, the spot of the second treatment laser beam (as well as the aiming light spot) is two-dimensionally scanned on the tissue of the patient's eye. The scanning part 230 also functions as a manipulator for changing the irradiation position of the spot of the second treatment laser beam.

In this embodiment, by synchronously repeating the start and stop of irradiation of the treatment laser beam from the treatment laser light source 211 and the start and stop of driving of the scanning part 230, a plurality of spots of the second treatment laser beam arranged in a predetermined scan pattern are formed on the tissue of the patient's eye.

### <Treatment Laser Irradiation Unit>

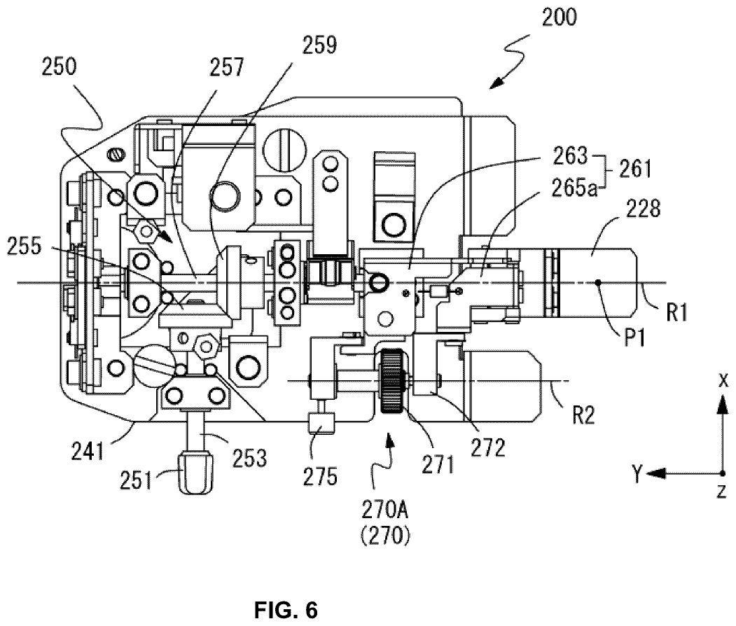

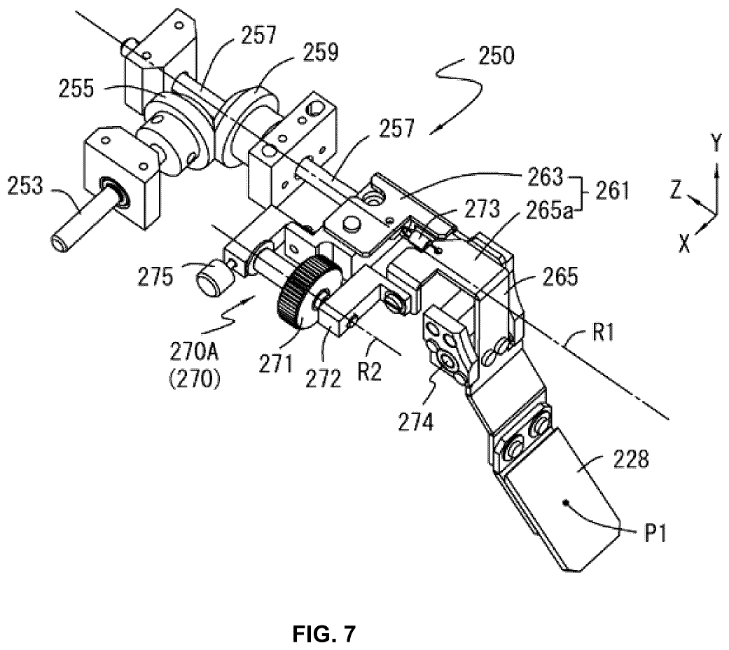

The structure of the treatment laser irradiation unit 200 is described with reference to FIGS. 4-8. FIG. 4 is a right-side sectional view of the unit's internal structure. FIG. 5 is a front view of an internal structure of the treatment laser irradiation unit 200 viewed from the patient's eye, where (a) shows a front view with the final mirror 228 at the irradiation position P1 and (b) is a front view with the mirror 228 at the retracted position P2. FIG. 6 is a top view of the internal structure. FIG. 7 is a perspective view of the mirror movement mechanism 250.

The treatment laser irradiation unit 200 mounted on the main body 100 comprises an optical element holder 220 (holding optical components from lens 221 to objective lens 227), a mounting part 240 (for attaching the unit to the main body), a mirror movement mechanism 250 (for moving the final mirror 228).

In FIG. 4, the base 241 of the unit 200 is mounted on the upper part of the main body 100 via a pedestal 242. A vertical support column 243, rotatably held inside the pedestal 242, fixes the base 241 thereon. The base 241 is adjustable in the left-right direction about the column's axis and secured by a fixing screw 244.

A barrel 220a (holding the objective lens 227 in FIG. 2) extending downward from the optical element holder 220 is supported by a support arm 247 extending forward (toward the patient's eye) from the base 241.

### <Mirror Movement Mechanism>

The mirror movement mechanism 250 is arranged on the base 241 and configured to move the final mirror 228 between the predetermined irradiation position P1 and retracted position P2. For example, the mechanism 250 displaces the final mirror 228 laterally (left or right relative to the operator) relative to the optical axis L1 of the objective lens 125 in the laser treatment apparatus main body 100, moving it to the retracted position P2. As shown in FIG. 4, the irradiation position P1 of the final mirror 228 is set on the optical axis L1 of the objective lens 125 in the main body 100, positioned in front of the objective lens 125. Furthermore, the irradiation position P1 is located between the split mirror 136 of the illumination optical system 130G and the objective lens 125. This configuration allows the second treatment laser light to be irradiated without increasing the working distance during its use compared to the working distance during irradiation of the first treatment laser light.

The final mirror 228, positioned at the irradiation location P1, is rotated about a rotation axis R1 parallel to the optical axis L1 of the objective lens 125, moving it to the lateral retracted position P2 (see (b) in FIG. 5). In this embodiment:

The rotation axis R1 is positioned above the objective lens 125.

The retracted position P2 is set above the objective lens 125.

Thus, the mirror movement mechanism 250 displaces the final mirror 228 from P1 to P2 laterally and above the objective lens. The rotation axis R1 is preferably aligned directly above (or substantially directly above) the optical axis L1 of the objective lens 125. While the rotation axis R1 may be offset laterally relative to L1, positioning it directly above L1 ensures stability, particularly when the optical component holder 220 (housing optical components from lens 221 to objective lens 227) is heavy.

For example, the mirror movement mechanism 250 comprises a lever 251 operated by the user. The lever 251 is positioned on the side of the base 241. When the lever 251 is operated, a lever rotation shaft 253 extending laterally relative to the rotation axis R1 is rotated. The lever rotation shaft 253 is rotatably supported by a block member fixed to the base 241. A bevel gear 255 is attached to the lever rotation shaft 253. A rotation shaft 257, centered on the rotation axis R1, is rotatably held by another block member fixed to the base 241. A bevel gear 259 is attached to the rotation shaft 257 and meshes with the bevel gear 255. When the lever 251 is operated, the lever rotation shaft 253 rotates, transmitting this rotation to the rotation shaft 257 via the bevel gears 255 and 259.

A mounting member 261 for holding the final mirror 228 is attached to the front side of the rotation shaft 257. The mounting member 261 comprises:

A first mounting member 263 positioned on the base 241, and

An arm 265 extending downward from the front side of the first mounting member 263 (downward when the final mirror 228 is at the irradiation position).

The final mirror 228 is attached to the lower tip of the arm 265. The arm 265 supports the final mirror 228 such that its center is positioned on the optical axis LS1 of the objective lens 227 while avoiding interference with the barrel part 220a extending downward from the optical component holder 220. In other words, when the final mirror 228 is at the irradiation position P1, the arm 265 has a shape extending above the mirror. Rotating the arm 265 about the rotation axis R1 displaces the final mirror 228 laterally relative to the objective lens 125.

The lower end of the barrel part 220a extending downward is positioned below or within a critical distance (i.e., a distance preventing upward retraction of the final mirror 228) from the upper end of the objective lens barrel 110a (see FIGS. 1 and 4) holding the objective lens 125 of the laser treatment apparatus main body 100. If the final mirror 228 were retracted upward directly from the irradiation position, it would interfere with the barrel part 220a. To resolve this, the present disclosure laterally displaces the final mirror 228 left or right relative to the objective lens 125 (optical axis L1) via the mirror movement mechanism 250, thereby avoiding interference with the barrel part 220a.

### <Final Mirror Size>

The size of the final mirror 228 is determined by balancing two competing requirements: Ensuring an adequate observation field of view via the observation optical system 140G.

Providing sufficient scanning range for the second treatment laser light scanned by the scanning part 230 of the second treatment laser irradiation optical system 200G.

A smaller mirror optimizes the observation field, while a larger mirror enhances the laser's scanning range. When the treatment laser irradiation unit 200 (equipped with the second laser optical system 200G) is mounted on the laser treatment apparatus main body 100 (which comprises the first treatment laser irradiation unit 110), the optical path of the observation system 140G incorporates an additional dichroic mirror 124 from the first treatment laser irradiation optical system 110G, compared to when mounted on a slit lamp (slit-lamp microscope) without the first laser system. This increases the optical path length, resulting in greater vignetting caused by the final mirror 228. To mitigate this, the final mirror 228 is made smaller than versions used with slit lamps.

For example, at a predetermined observation magnification (e.g., 12.5×, frequently used) of the variable magnification optical system 142 in the observation optical system 140G, the size of the final mirror 228 is set to:
Ensure at least 80% of the observation field of view is maintained, and
Permit irradiation of the second treatment laser light in a predetermined scan pattern (on fundus tissue) via scanning by the scanning part 230, within the observable field range.

### <Adjustment Mechanisms>

The treatment laser irradiation unit 200 includes an adjustment mechanism 270 configured to adjust the tilt angle of the final mirror 228 relative to the optical axis L1 of the objective lens 125. The adjustment mechanism 270 includes a vertical adjustment mechanism 270A and a horizontal adjustment mechanism 270B. The vertical adjustment mechanism 270A configured to adjust the mirror's pitch angle via a rotating knob 271, which moves a member 272 connected to the arm 265. A tension spring 273 maintains alignment, and a fixing screw 275 locks the adjusted position. The horizontal adjustment mechanism 270B: Adjusts the mirror's yaw angle by rotating the base 241 about the support column 243.

The vertical adjustment mechanism 270A is attached to the first mounting member 263 and moves with the final mirror 228 via the mirror movement mechanism 250. The vertical adjustment mechanism 270 comprises a rotation knob 271 rotatable about an axis R2 parallel to the rotation axis R1. Rotating the knob 271 moves a moving member 272, engaged with the knob's feed screw, axially along axis R2. The rear end 265a of the arm 265 is connected to the moving member 272. The arm 265 is pivotable about a rotation support part 274 located at the front of the first mounting member 263. A tension spring 273 is arranged between the first mounting member 263 and the rear end 265a of the arm 265, applying a rearward force to the arm. When the rotation knob 271 is rotated clockwise or counterclockwise, the moving member 272 shifts forward or backward, causing the arm 265 to pivot about the rotation support part 274. This adjusts the vertical tilt angle of the final mirror 228 attached to the tip of the arm 265. Once adjusted, a fixing screw 275 near the rotation knob 271 is tightened to secure both the knob's position and the mirror's tilt angle.

As shown in FIG. 4, the horizontal adjustment mechanism 270B comprises a support column 243 rotatably held inside the pedestal 242 and a fixing screw 244. When the fixing screw 244 is loosened, the base 241 fixed to the support column 243 is rotated left or right about the column's axis, adjusting the horizontal tilt angle of the final mirror 228 mounted on the base 241.

### <Final Mirror Position Detection Mechanism>

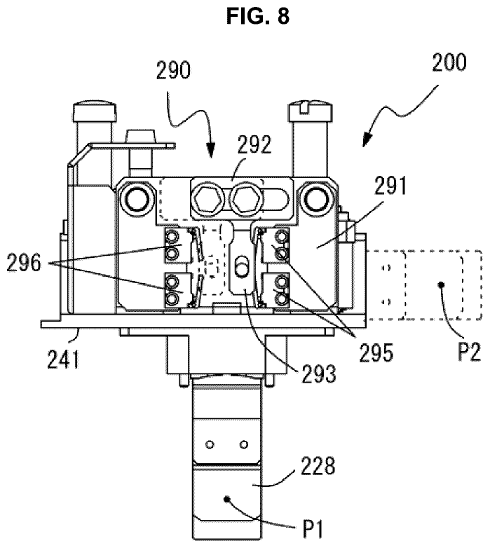

The treatment laser irradiation unit 200 includes a final mirror position detection mechanism 290 configured to detect whether the final mirror 228 is moved to the irradiation position P1 or the retracted position P2. The final mirror position detection mechanism 290 is disposed on the base 241 of the mirror movement mechanism 250. FIG. 8 is a back side view of the final mirror position detection mechanism 290. In FIG. 8, the final mirror 228 is at the irradiation position P1.

In FIG. 8, a slide plate 292 is disposed on a rear plate 291 of the base 241 such that it can slide in the X-direction (left-right direction). The slide plate 292 is linked to the rotation of the rotation shaft 257 (for example, via a mechanism such as a cam that converts rotational motion into linear motion), and moves in the left-right direction as shown in FIG. 8. On both sides of an actuating plate 293 extending downward from the slide plate 292, a first detector 295 and a second detector 296, such as microswitches, are arranged. In this embodiment, two first detectors 295 and two second detectors 296 are provided, respectively. If one of the two first detectors 295 fails, the movement position of the final mirror 228 can still be detected by the other.

When the final mirror 228 is moved to the irradiation position P1, the slide plate 292 and actuating plate 293 are displaced toward the first detector 295, and this movement is detected by the first detector 295. Conversely, when the final mirror 228 is moved to the retracted position P2, as indicated by the dashed line in FIG. 8, the slide plate 292 and actuating plate 293 are displaced toward the second detector 296, and this movement is detected by the second detector 296.

### <Housing for the Final Mirror>

As shown in (b) of FIG. 5, the treatment laser irradiation unit 200 comprises a housing compartment 280 that accommodates the final mirror 228 within a cover when the mirror is moved to the retracted position P2. The retracted position P2 is set laterally and above the objective lens barrel 110a, which holds the objective lens 125. Therefore, the housing compartment 280 is provided on the lateral side of the barrel part 220a. For example, the housing compartment 280 may be formed integrally within the cover of the treatment laser irradiation unit 200. This housing compartment 280 prevents accidental contact with the final mirror 228 by the operator during treatment with the first treatment laser light and also helps to reduce the accumulation of dust or contamination on the final mirror 228.

### <Control System>

The control unit 50 governs the overall control of the ophthalmic laser treatment apparatus 1. For example, the control unit 50 comprises a CPU having at least one processor and at least one circuit, RAM, ROM, non-volatile memory, and other components. Then, at least one of the circuit and the processor is configured to cause the control unit 50 to perform several functions and processes as described herein.

In the present disclosure, the term “processor” may refer to a single hardware processor or several hardware processors that are configured to execute computer program code (i.e., one or more instructions of a program). In other words, a processor may be one or more programmable hardware devices. For instance, a processor may be a general-purpose or embedded processor and include, but not necessarily limited to, CPU (a Central Processing Circuit), a microprocessor, a microcontroller, and PLD (a Programmable Logic Device) such as FPGA (a Field Programmable Gate Array).

The term “memory” in the present disclosure may refer to a single or several hardware memory configured to store computer program code (i.e., one or more instructions of a program) and/or data accessible by a processor. A memory may be implemented using any suitable memory technology, such as static random-access memory (SRAM), synchronous dynamic RAM (SDRAM), nonvolatile/Flash-type memory, or any other type of memory. Computer program code may be stored on the memory and, when executed by a processor, cause the processor to perform the above-described various functions.

In the present disclosure, the term “circuit” may refer to a single hardware logical circuit or several hardware logical circuits (in other words, “circuitry”) that are configured to perform one or more functions. In other words (and in contrast to the term “processor”), the term “circuit” refers to one or more non-programmable circuits. For instance, a circuit may be IC (an Integrated Circuit) such as ASIC (an application-specific integrated circuit) and any other types of non-programmable circuits.

In the present disclosure, the phrase “at least one of (i) a circuit and (ii) a processor” should be understood as disjunctive (logical disjunction) where the circuit and the processor can be optional and not be construed to mean “at least one of a circuit and at least one of a processor”. Therefore, in the present disclosure, the phrase “at least one of a circuit and a processor is configured to cause the control unit to perform functions” should be understood as “only the circuit can cause the control unit to perform all the functions”. Further, the phrase “at least one of a circuit and a processor is configured to cause the control unit to perform functions” should be understood as “only the processor can cause the control unit to perform all the functions”. Moreover, the phrase “at least one of a circuit and a processor is configured to cause the control unit to perform functions” should be understood as “the circuit can cause the control unit to perform at least one of the functions and the processor can cause the control unit to perform the remaining functions”. In the last example, if the control unit performs functions A to C, for example, the functions A and B among the functions A to C may be implemented by a circuit, while the remaining function C may be implemented by a processor.

The control unit 50 is connected to electrical elements of the laser treatment apparatus main body 100 and the treatment laser irradiation unit 200 (e.g., laser sources, illumination sources, detectors, scanning parts) and controls their operations. Additionally, the control unit 50 is connected to:
The first control box 51,
The second control box 52,
The 3D mouse 53,
The foot switch 54, and
The storage unit 55.

The control unit 50 may also function as a receiver for signals input from these components. Furthermore, it serves as a display controller, managing the screens of the display 51a on the first control box 51 and the display 52a on the second control box 52. The control unit 50 updates the displays based on detection results from the final mirror position detection mechanism 290.

Control functions of the control unit 50 may be distributed between the first and second control boxes 51/52. This allows flexible configuration of the apparatus 1, where the main body 100 is installed first and the irradiation unit 200 is added later.

The first control box 51 comprises a display 51a and allows for various settings related to the laser irradiation conditions of the laser treatment apparatus main body 100. Similarly, the second control box 52 comprises a display 52a and allows for various settings related to the laser irradiation conditions of the treatment laser irradiation unit 200. The first control box 51 and the second control box 52 may also be integrated into a single unit and used commonly for both the laser treatment apparatus main body 100 and the treatment laser irradiation unit 200.

### <Operation>

The operation of the ophthalmic laser treatment apparatus 1 is described separately for the laser treatment apparatus main body 100 and the treatment laser irradiation unit 200.

### <Treatment Operation by Laser Treatment Apparatus Main Body>

When using the laser treatment apparatus main body 100, the final mirror 228 is positioned at the retracted position P2. When the second detector 296 detects the mirror's retracted position, the control unit 50 enables laser irradiation settings on the display 51a of the first control box 51.

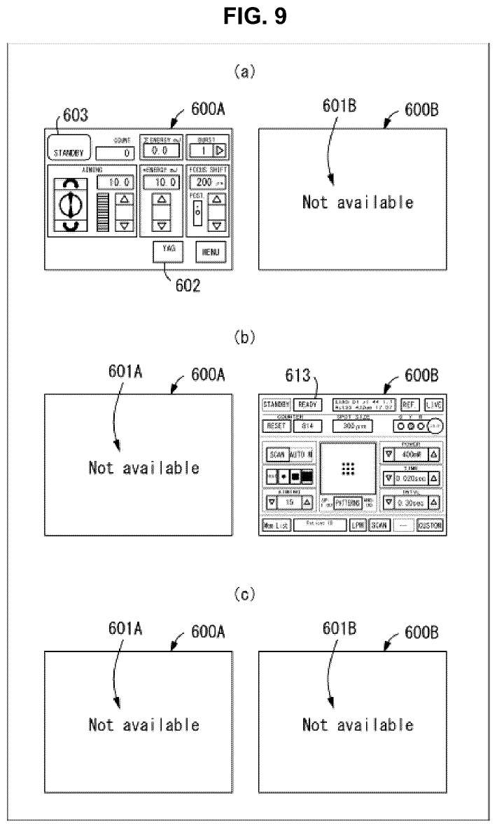

FIG. 9 illustrates examples of screen 600A displayed on the display 51a of the first control box 51 and screen 600B displayed on the display 52a of the second control box 52 during use of the laser treatment apparatus main body 100. When the final mirror 228 is at the retracted position P2, laser irradiation by the main body 100 is enabled. Based on this detection, screen 600A on the first control box 51 displays settings for configuring the laser irradiation conditions of the main body 100, as shown in (a) of FIG. 9. Conversely, screen 600B on the second control box 52 displays a message 601B indicating that the treatment laser irradiation unit 200 is unavailable (or that the main body 100 is in use). This allows the operator to easily identify which control box is active and configure the laser irradiation conditions of the main body 100 appropriately.

The laser treatment apparatus main body 100 offers two modes:

YAG Mode: Uses a 1064 nm YAG laser to generate micro-plasma for incising tissues such as the posterior capsule or iris.

SLT Mode: Converts the YAG laser to 532 nm (green) for selective targeting of pigmented cells in the trabecular meshwork.

These modes are selected via a switch 602 on the screen 600A. The following describes the YAG mode.

In YAG mode, the operator sets irradiation conditions on the screen 600A, including: Energy level of the treatment laser light,
Focus shift of the laser beam,
Light intensity of the aiming beam,
Number of pulses emitted per irradiation (in BURST mode).

After aligning the aiming light with the affected area using the joystick 106, the operator switches from STANDBY to READY via switch 603 on the screen 600A. Pressing the foot switch 54 then activates the treatment laser light source 111, irradiating the target tissue via the first treatment laser irradiation optical system 110G.

### <Treatment Operation by Treatment Laser Irradiation Unit>

When performing laser irradiation using the treatment laser irradiation unit 200, the operator manipulates the lever 251 to move the final mirror 228 from the retracted position P2 to the irradiation position P1. When the first detector 295 detects that the final mirror 228 is at the irradiation position P1, the screen 600B on the second control box 52 switches to a screen for setting laser irradiation conditions for the treatment laser irradiation unit 200, as shown in (b) of FIG. 9. Conversely, when the second detector 296 detects that the final mirror 228 has moved from the retracted position P2, the screen 600A on the first control box 51 displays a message 601A indicating that the laser treatment apparatus main body 100 is unavailable (or that the treatment laser irradiation unit 200 is in use). If the final mirror 228 is not correctly positioned at either the irradiation position P1 or the retracted position P2, both screens 600A and 600B display messages 601A and 601B indicating unavailability, as shown in (c) of FIG. 9. This allows the operator to recognize which control box is active and appropriately configure the laser irradiation conditions for the treatment laser irradiation unit 200.

On the screen 600B in (b) of FIG. 9, laser irradiation conditions for photocoagulation treatment via the treatment laser irradiation unit 200 are set. These conditions comprise:
Spot size of the treatment laser light,
Energy level of the treatment laser light,
Coagulation time,
Interval time for moving the spot position,
Light intensity of the aiming beam.

The treatment laser irradiation unit 200 can form multiple spots arranged in a predetermined scan pattern by scanning the treatment laser beam with the scanning part 230 in response to a single trigger signal. Therefore, the scan pattern of the treatment laser spots can also be configured as a laser irradiation condition. For example, scan patterns such as:

Square type: Multiple spots (e.g., 3×3 grid of 9 spots) arranged in a square.

Triangle type: Multiple spots arranged in a triangular shape.

Arc type: Multiple spots arranged in an arc.

Predefined scan patterns can be registered in advance and stored in the storage unit 55. These registered patterns can be retrieved and set via operation of the 3D mouse (described later).

After configuring the laser irradiation conditions for the treatment laser irradiation unit 200, the operator observes the affected area of the patient's eye, illuminated by the illumination unit 130, through the observation optical system 140G and aligns the aiming light with the target tissue. Upon switching from STANDBY to READY via switch 613 on the screen 600B, the treatment laser light becomes ready for emission. When the foot switch 54 is pressed, the treatment laser light source 211 is activated in response to the trigger signal. The treatment laser light emitted from the source 211 is guided by the second treatment laser irradiation optical system 200G, reflected by the final mirror 228, and irradiated onto the affected area (fundus tissue) of the patient's eye. If a scan pattern is set, the spots of the treatment laser light are scanned across the tissue according to the selected pattern.

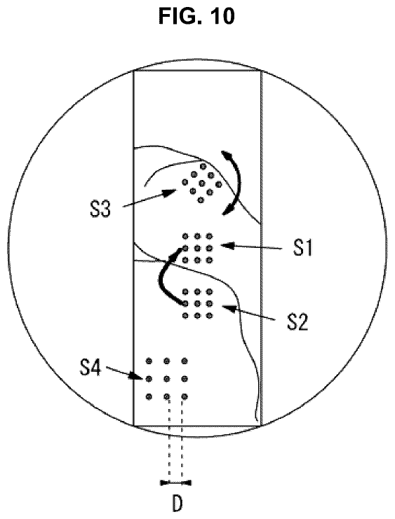

FIG. 10 illustrates examples of scan patterns in which spots of the treatment laser light are arranged. In this example, the scanning part 230 sequentially scans the spots of the treatment laser light to form a 3×3 grid of nine spots at irradiation position S1.

In photocoagulation treatment, the treatment laser light is irradiated over a wide area of the fundus tissue. Conventionally, the irradiation position was adjusted by manually moving the final mirror in front of the objective lens using a mechanical manipulator mechanism. However, when a treatment laser irradiation unit 200 equipped with a scanning part 230 is mounted, mechanically adjusting the final mirror 228 (which moves between the irradiation and retracted positions) becomes impractical.

To address this, the present disclosure employs a 3D mouse 53 (an example of an operation unit) in conjunction with the scanning part 230. The 3D mouse 53 functions as an electronic manipulator, enabling two-dimensional displacement of the irradiation position (spot position) of the second treatment laser light on the patient's eye tissue.

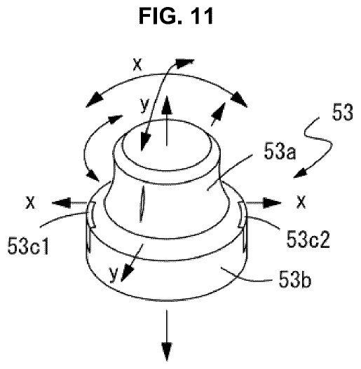

FIG. 11 illustrates the configuration of the 3D mouse 53 and the input of operation signals. The 3D mouse 53 comprises an operation member 53a, which is gripped by the operator's hand or fingers, and a base part 53b. The operation member 53a is tiltable in two dimensions relative to the base part 53b-specifically, in the x-direction (left-right) and y-direction (front-back). Tilting the member in the four directions (left, right, forward, backward) inputs four operation signals: left tilt signal, right tilt signal, forward tilt signal, and backward tilt signal. Additionally, the operation member 53a is slidable in two dimensions (x- and y-directions) relative to the base part 53b. Sliding the member in the four directions inputs four operation signals: left slide signal, right slide signal, forward slide signal, and backward slide signal.

Additionally, the operation member 53a can be rotated left or right relative to the base part 53b, allowing for the input of two operation signals: a left rotation signal and a right rotation signal, depending on the direction of rotation. The operation member 53a can also be moved upward or downward relative to the base part 53b, enabling the input of two more operation signals: an upward movement signal and a downward movement signal. Furthermore, the base part 53b is equipped with a left button 53c1 and a right button 53c2, each of which can be operated to input a left button signal and a right button signal, respectively.

In the 3D mouse 53, which can input multiple operation signals, the manipulator function signal (for two-dimensionally moving the irradiation position of the second treatment laser light on the patient's eye tissue) is assigned to the left tilt signal, right tilt signal, forward tilt signal, and backward tilt signal generated by tilting the operation member 53a in the four directions (left, right, forward, backward). Specifically:

When a left tilt signal or right tilt signal is input, the control unit 50 controls the scanning part 230 to displace the irradiation position (spot) of the second treatment laser light on the tissue in the X direction.

When a forward tilt signal or backward tilt signal is input, the control unit 50 controls the scanning part 230 to displace the irradiation position (spot) in the Y direction.

For example, when the manipulator function signal is input during a scan pattern, the irradiation position shifts from S1 to S2 in FIG. 10.

In some cases, operators may find the tilt operation of the operation member 53a challenging when inputting manipulator function signals. To address this, the present embodiment assigns manipulator function signals to the slide operations of the operation member 53a in four directions: left, right, forward, and backward. Specifically: Inputting left slide, right slide, forward slide, or backward slide signals controls the scanning part 230 to displace the irradiation position (spot) of the second treatment laser light in the XY directions.

In other words, both tilt and slide operation signals from the 3D mouse 53 are accepted by the control unit 50 as manipulator function signals for two-dimensional movement of the irradiation position. This allows:

Operators uncomfortable with tilt operations (first operation) to use slide operations (second operation).

Operators uncomfortable with slide operations to use tilt operations.

Thus, the ophthalmic laser treatment apparatus 1 facilitates XY displacement signal input, significantly enhancing the usability of the treatment laser irradiation unit 200.

In the 3D mouse 53 of the present embodiment, at least one of the upward movement signal or downward movement signal generated by vertically moving the operation member 53a (an example of a third operation) may be assigned as the manipulator function signal to return the irradiation position to the origin. For example, when the upward or downward movement signal is input from the operation member 53a and received by the control unit 50, the scanning part 230 is controlled to return the irradiation position from S2 to S1 in FIG. 10.

Additionally, the 3D mouse 53 in this embodiment is shared for both the manipulator function and the pattern-setting function, which configures the scan pattern of the second treatment laser light spots. For instance:

Left/right rotation signals from the operation member 53a are assigned to rotate the scan pattern.

These signals are accepted by the control unit 50.

As shown in FIG. 10, the scan pattern at irradiation position S3 is an example of the pattern at S1 rotated 90 degrees left or right.

For example, the signals from pressing the left button 53c1 and right button 53c2 are assigned to modify (increase or decrease) the interval D between adjacent spots. In FIG. 10, the scan pattern at irradiation position S4 illustrates an example where the interval D between spots is increased compared to the pattern at S1.

The pattern-setting functions described above—rotating the scan pattern and adjusting the interval D—are merely examples. Other scan pattern configurations may be assigned to various operations of the 3D mouse 53. For instance, the upward movement signal of the operation member 53a could be assigned to sequentially recall and set predefined scan pattern types (e.g., square, triangle, arc) stored in the storage unit 55. The assignment of operation signals for the 3D mouse 53 can be customized via a designated settings screen on the control box 52.

By integrating both manipulator and pattern-setting functions into the 3D mouse 53, the operator can precisely irradiate the patient's eye while viewing through the microscope 141 of the observation optical system 140G, without needing to divert their gaze. This dual functionality significantly enhances the usability of the treatment laser irradiation unit 200.

### <Modifications>

While the above embodiment describes the mirror movement mechanism 250 rotating the final mirror 228 about the rotation axis R1 to move it laterally to the retracted position P2, alternative configurations are possible. For example, the mechanism 250 may first linearly displace the final mirror 228 laterally relative to the optical axis of the objective lens 125 (to avoid interference with the barrel 220a) before retracting it upward via linear or rotational motion to position P2.

1. An ophthalmic laser treatment apparatus, comprising:
a laser treatment apparatus main body configured to irradiate a first treatment laser light onto a patient's eye via an objective lens;
a treatment laser light irradiation unit mounted above the objective lens on the laser treatment apparatus main body, the treatment laser light irradiation unit configured to irradiate a second treatment laser light, different from the first treatment laser light, onto the patient's eye via a final mirror disposed at a predetermined irradiation position in front of the objective lens; and
a movement mechanism configured to move the final mirror between the irradiation position and a predetermined retracted position outside an optical path of the first treatment laser light, wherein
the movement mechanism is further configured to laterally move the final mirror from the irradiation position to the retracted position relative to the objective lens.

2. The ophthalmic laser treatment apparatus according to claim 1, wherein
the movement mechanism is further configured to move the final mirror from the irradiation position to the retracted position by pivoting the final mirror about a rotation axis parallel to an optical axis of the objective lens.

3. The ophthalmic laser treatment apparatus according to claim 2, wherein
the rotation axis is positioned above the objective lens,
the retracted position is set above the objective lens, and
the movement mechanism is configured to move the final mirror from the irradiation position to the retracted position laterally and above the objective lens.

4. The ophthalmic laser treatment apparatus according to claim 1, further comprising
a vertical adjustment mechanism configured to adjust a tilt angle of the final mirror in an up-down direction, wherein
the vertical adjustment mechanism is moved together with the final mirror by the movement mechanism.

5. The ophthalmic laser treatment apparatus according to claim 1, wherein
a lower end of an irradiation barrel of an irradiation optical system for the second treatment laser light is positioned (i) in front of the objective lens and (ii) below or within a predetermined distance from an upper end of an objective lens barrel holding the objective lens.

6. The ophthalmic laser treatment apparatus according to claim 1, further comprising:
an observation optical system configured to observe the patient's eye via the objective lens; and
an illumination optical system configured to project illumination light onto the patient's eye via a reflective member disposed on a patient's eye side of the objective lens, wherein
the irradiation position is set between the objective lens and the reflective member.

7. The ophthalmic laser treatment apparatus according to claim 1, wherein
the treatment laser irradiation unit comprises a housing at a position lateral to a barrel of an irradiation optical system for the second treatment laser light positioned above the final mirror, and
the housing is configured to accommodate the final mirror moved to the retracted position within a cover.

8. The ophthalmic laser treatment apparatus according to claim 1, further comprising:
a detection sensor configured to detect whether the final mirror is at the irradiation position or the retracted position; and
a control unit configured to control irradiation of the first treatment laser light and the second treatment laser light based on a detection result from the detection sensor.

9. The ophthalmic laser treatment apparatus according to claim 1, further comprising:
an operation unit configured to input an operation signal from an operator; and
a control unit, wherein
an irradiation optical system for the second treatment laser light comprises a scanning part configured to scan the second treatment laser light to form a plurality of spots arranged in a predetermined scan pattern,
the operation unit is further configured to perform both (i) a manipulator function of inputting a movement signal for two-dimensionally displacing an irradiation position of the second treatment laser light on the patient's eye tissue and (ii) a pattern setting function of setting the scan pattern, and
the control unit is configured to control the scanning part based on signals of the manipulator function and the pattern setting function that are input from the operation unit.

10. An ophthalmic laser treatment apparatus, comprising:
a laser treatment apparatus main body configured to irradiate a first treatment laser light onto a patient's eye via an objective lens;
a treatment laser irradiation unit mounted on the laser treatment apparatus main body, the treatment laser irradiation unit comprising a second treatment laser irradiation optical system with a scanning part configured to scan a second treatment laser light, different from the first treatment laser light, onto a patient's eye tissue via a final mirror disposed at a predetermined irradiation position in front of the objective lens;
a movement mechanism configured to move the final mirror between the irradiation position and a retracted position outside an optical path of the first treatment laser light;
an operation unit configured to input an operation signal from an operator; and
a control unit, wherein
the second treatment laser irradiation optical system is configured to form a plurality of spots arranged in a predetermined scan pattern via the scanning part,
the operation unit is further configured to perform both (i) a manipulator function of inputting a movement signal for two-dimensionally displacing an irradiation position of the second treatment laser light on the patient's eye tissue and (ii) a pattern setting function of setting the scan pattern, and
the control unit is configured to control the scanning part based on signals of the manipulator function and the pattern setting function that are input from the operation unit.

11. The ophthalmic laser treatment apparatus according to claim 10, wherein
the operation unit comprises an operation member operable in a first two-dimensional operation and a second two-dimensional operation distinct from the first operation, and
regardless of whether a signal of the first operation and a signal of the second operation is input, the control unit is further configured to accept the signal as the signal of the manipulator function.

12. The ophthalmic laser treatment apparatus according to claim 11, wherein
the operation member is further operable in a third operation distinct from the first and second operations, and
the control unit is configured to receive the signal of the manipulator function for returning the irradiation position of the second treatment laser light to an origin in response to receiving a signal of the third operation from the operation member.

13. The ophthalmic laser treatment apparatus according to claim 10, wherein
the pattern setting function includes at least one of a first setting for rotating the scan pattern, a second setting for adjusting spacing between the plurality of spots of the scan pattern, and a third setting for modifying arrangement of the plurality of spots, and
the signal of the pattern setting function is input via an operation distinct from the manipulator function by an operation member of the operation unit.

14. The ophthalmic laser treatment apparatus according to claim 10, wherein
the operation unit is a 3D mouse configured to input a plurality of operation signals via hand or finger manipulation.

15. The ophthalmic laser treatment apparatus according to claim 10, further comprising
an observation optical system configured to observe the patient's eye via the objective lens, wherein
the final mirror, when at the irradiation position in front of the objective lens, ensures at least 80% of an observation field of view in the observation optical system and is sized to enable irradiation of the second treatment laser light in the predetermined scan pattern.

16. The ophthalmic laser treatment apparatus according to claim 10, further comprising
an illumination optical system configured to project illumination light onto the patient's eye via a reflective member disposed on a patient's eye side of the objective lens, wherein
the irradiation position is set between the objective lens and the reflective member.

17. A treatment laser irradiation unit mounted above an objective lens of a laser treatment apparatus main body configured to irradiate a first treatment laser light onto a patient's eye via the objective lens, the treatment laser irradiation unit comprising:
a second treatment laser irradiation optical system configured to irradiate a second treatment laser light, different from the first treatment laser light, onto the patient's eye via a final mirror disposed at a predetermined irradiation position in front of the objective lens; and
a movement mechanism configured to move the final mirror between the irradiation position and a retracted position outside an optical path of the first treatment laser light, wherein
the movement mechanism is configured to move the final mirror to the retracted position by laterally moving the final mirror at the irradiation position relative to the objective lens.

18. The treatment laser irradiation unit according to claim 17, wherein
the movement mechanism is configured to move the final mirror from the irradiation position to the retracted position by pivoting the final mirror about a rotation axis parallel to an optical axis of the objective lens.

19. The treatment laser irradiation unit according to claim 17, further comprising:
an operation unit configured to input an operation signal from an operator; and
a control unit, wherein
the second treatment laser irradiation optical system comprises a scanning part configured to scan the second treatment laser light on a patient's eye tissue to form a plurality of spots arranged in a predetermined scan pattern,
the operation unit is further configured to perform both (i) a manipulator function of inputting a movement signal for two-dimensionally displacing an irradiation position of the second treatment laser light on the patient's eye tissue and (ii) a pattern setting function of setting the scan pattern, and
the control unit is configured to control the scanning part based on signals of the manipulator function and the pattern setting function that are input from the operation unit.
